(19) United States
(12) Patent Application Publication
(10) Pub. No.: US 2009/0306167 A1
(43) Pub. Date: Dec. 10, 2009

(54) Pharmaceutical Compositions for Intranasal Administration Comprising a Melatonin Receptor Agonist, Uses Thereof

(76) Inventor: Edwin Cohen, Mahway, NJ (US)

Correspondence Address: GOODWIN PROCTER LLP;PATENT ADMINISTRATOR, 53 STATE STREET, EXCHANGE PLACE, BOSTON, MA, 02109-2881, US

(21) Appl. No.: 12/293,197

(22) PCT Filed: Mar. 16, 2007
(86) PCT No.: PCT/US07/06657

§ 371 Date: Feb. 18, 2009

Related U.S. Application Data
(60) Provisional application No. 60/782,761, filed on Mar. 16, 2006.

(51) Int. Cl. A61K 31/404; A61K 31/343; A61K 31/335; A61P 25/00

(52) U.S. Cl. 514/411; 514/468; 514/452

(57) ABSTRACT

The present invention relates to intranasally deliverable compositions comprising melatonin receptor agonists and to methods of using such compositions in the treatment of various diseases and disorders.

### RELATED APPLICATIONS

This application claims the benefit of priority to U.S. Provisional Patent Application Ser. No. 60/782,761, filed Mar. 16, 2006, the contents of which are hereby incorporated by reference.

### FIELD OF THE INVENTION

The present invention relates to pharmaceutical compositions comprising a melatonin receptor agonist and to methods of using such compositions to treat and/or prevent various diseases and disorders.

### BACKGROUND OF THE INVENTION

Melatonin (N-acetyl-5-methoxytryptamine), a hormone synthesized and secreted principally in the pineal gland, exerts suppressive effects on pigment cells and the female gonads, and acts as a synchronous factor of biological clock while taking part in transmittance of photoperiodic code. Therefore, melatonin is expected to be used for the therapy of diseases related with melatonin activity, such as reproduction and endocrinic disorders, sleep-awake rhythm disorders, jet-lag syndrome and various disorders related to aging, etc.

As previously reported, however, melatonin is easily metabolized by metabolic enzymes in vivo. Therefore, it is doubtful whether melatonin is suitable as a pharmaceutical substance. Pharmaceutically acceptable compounds and formulations which have agonistic or antagonistic activity towards melatonin receptors are therefore desired. If suitable formulations of melatonin receptor agonists/antagonists could be provided, a significant advance in the art would result.

### SUMMARY OF THE INVENTION

In various embodiments, the present invention provides intranasal compositions comprising melatonin receptor agonists (also called “melatonin agonists”) and/or antagonists and methods for using the same in treatment and/or prevention of various diseases and disorders. In one embodiment, the melatonin agonists are tricyclic compounds.

In another embodiment, the present invention provides a pharmaceutical composition for intranasal administration to a mammal comprising a therapeutically effective amount of a melatonin agonist, a liquid nasal carrier, and optionally one or more pharmaceutically acceptable excipients.

The related terms “therapeutically effective amount,” “prophylactically effective amount,” or “effective amount” as used herein refer to an amount of drug or agent that is sufficient to elicit the required or desired therapeutic and/or prophylactic response, as the particular treatment context may require.

In another embodiment, the present invention provides a method of treating a mammal comprising intranasally administering to the mammal an effective amount of a composition as described herein. In a related embodiment, the mammal suffers from a reproductive, endocrine, sleep-related, jet lag-related or aging disease or disorder.

In still another embodiment, the present invention provides an intranasal unit-dose delivery device comprising one or more sealed vessels or containers comprising a sterilized, pharmaceutical composition as described herein. In a related embodiment, upon positioning the device a fixed distance away from a detection laser beam, actuating the device to produce a spray plume perpendicular to the laser beam, and detecting droplet size distribution of the spray plume with the laser beam, the spray plume has defined droplet size dispersion characteristics.

In another embodiment, upon positioning the above device a fixed distance away from an impaction plate, actuating the device to produce a spray pattern onto the impaction plate, and measuring the diameter of the spray pattern, the spray pattern has a defined maximum diameter, minimum diameter and/or span.

These and other embodiments of the present invention are described in more detail herein below.

### DETAILED DESCRIPTION OF THE INVENTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any way. Embodiments illustrated under any heading may be combined with embodiments illustrated under any other heading.

The use of numerical values in the various ranges specified in this application, unless expressly indicated otherwise, are stated as approximations as though the minimum and maximum values within the stated ranges were both preceded by the word “about.” In this manner, slight variations above and below the stated ranges can be used to achieve substantially the same results as values within the ranges. As used herein, the terms “about” and “approximately” when referring to a numerical value shall have their plain and ordinary meanings to one skilled in the art of pharmaceutical sciences or the art relevant to the range or element at issue. The amount of broadening from the strict numerical boundary depends upon many factors. For example, some of the factors to be considered may include the criticality of the element and/or the effect a given amount of variation will have on the performance of the claimed subject matter, as well as other considerations known to those of skill in the art. Thus, as a general matter, “about” or “approximately” broaden the numerical value. For example, in some cases, “about” or “approximately” may mean ±5%, or ±10%, or ±20%, or ±30% depending on the relevant technology. Also, the disclosure of ranges is intended as a continuous range including every value between the minimum and maximum values recited.

It is also to be understood that any ranges, ratios, and ranges of ratios that can be formed by any of the numbers or data present herein represent further embodiments of the present invention. This includes ranges that can be formed that do or do not include a finite upper and/or lower boundary. Accordingly, the skilled person will appreciate that such ratios, ranges and values are unambiguously derivable from the data presented herein.

### Melatonin Agonists

Compositions of the invention comprise at least one pharmaceutically acceptable melatonin agonist. The term “melatonin agonist” as used herein includes any substance, naturally or synthetically derived, that bind to the melatonin receptor in an agonistic manner. In one embodiment, the melatonin agonist is a tricyclic compound.

In another embodiment, the melatonin agonist comprises a compound which has an R¹—CO-amino-C₁₋₄ alkylene group (in which R¹ has the meanings as defined hereinafter) at Y of the basic skeleton moiety of the Formula I:

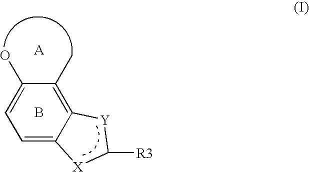

wherein all symbols have the meanings as defined hereinafter.

In another embodiment, the melatonin agonist is represented by Formula II:

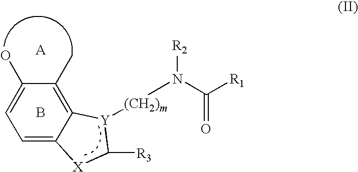

wherein R¹ represents an optionally substituted hydrocarbon group, an optionally substituted amino group or an optionally substituted heterocyclic group; R² represents a hydrogen atom or an optionally substituted hydrocarbon group; R³ represents a hydrogen atom, an optionally substituted hydrocarbon group or an optionally substituted heterocyclic group; X represents CHR⁴, NR⁴, O or S in which R⁴ represents a hydrogen atom or an optionally substituted hydrocarbon group; Y represents C, CH or N, provided that when X is CH₂, Y is C or CH;

[symbol] independently represents a single bond or a double bond;

ring A represents an optionally substituted, 5- to 7-membered oxygen-containing heterocyclic ring;

ring B represents an optionally substituted benzene ring; and m represents an integer of 1 to 4; or a salt thereof (hereinafter collectively referred to as Formula II).

(1) In one embodiment, the melatonin agonist is of Formula II.

(2) In another embodiment, the melatonin agonist is of Formula II wherein R¹ is (i), (ii) or (iii) as set forth below wherein:

(i) is a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkylcarbamoyl, di-C₁₋₆ alkylcarbamoyl, C₆₋₁₀ aryl-carbamoyl, C₆₋₁₀ aryl, C₆₋₁₀ aryloxy and an optionally halogenated C₁₋₆ alkyl-carbonylamino;

(ii) is an amino group which may be substituted by 1 or 2 substituents selected from the group consisting of a C₁₋₆ alkyl, C₂₋₆ alkenyl, C₂₋₆ alkynyl, C₃₋₆ cycloalkyl and C₆₋₁₄ aryl group, each of which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino, and

(iii) is a 5- to 14-membered heterocyclic group containing, besides carbon atoms, 1 to 3 hetero atoms selected from nitrogen atom, oxygen atom and sulfur atom, which group may be substituted by 1 to 5 substituents selected from the group consisting of a halogen, C₁₋₆ alkyl, C₃₋₆ cycloalkyl, C₂₋₆ alkynyl, C₂₋₆ alkenyl, C₇₋₁₁ aralkyl, C₆₋₁₀ aryl, C₁₋₆ alkoxy, C₆₋₁₀ aryloxy, formyl, C₁₋₆ alkyl-carbonyl, C₆₋₁₀ aryl-carbonyl, formyloxy, C₁₋₆ alkyl-carbonyloxy, C₆₋₁₀ arylcarbonyloxy, carboxyl, C₁₋₆ alkoxy-carbonyl, C₇₋₁₁ aralkyloxy-carbonyl, carbamoyl, an optionally halogenated C₁₋₄ alkyl, oxo, amidino, imino, amino, mono-C₁₋₄ alkylamino, di-C₁₋₄ alkylamino, 3- to 6-membered cyclic amino, C₁₋₃ alkylenedioxy, hydroxy, nitro, cyano, mercapto, sulfo, sulfino, phosphono, sulfamoyl, mono-C₁₋₆ alkylsulfamoyl, di-C₁₋₆ alkylsulfamoyl, C₁₋₆ alkylthio, C₆₋₁₀ arylthio, C₁₋₆ alkylsulfinyl, C₆₋₁₀ arylsulfinyl, C₁₋₆ alkylsulfonyl and C₆₋₁₀ arylsulfonyl;

R² is (i) a hydrogen atom or (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino;

R³ is (i) a hydrogen atom, (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino or (iii) a 5- to 14-membered heterocyclic group containing, besides carbon atoms, 1 to 3 hetero atoms selected from nitrogen atom, oxygen atom and sulfur atom, which group may be substituted by 1 to 5 substituents selected from the group consisting of a halogen, C₁₋₆ alkyl, C₃₋₆ cycloalkyl, C₂₋₆ alkynyl, C₂₋₆ alkenyl, C₇₋₁₁ aralkyl, C₆₋₁₀ aryl, C₁₋₆ alkoxy, C₆₋₁₀ aryloxy, formyl, C₁₋₆ alkyl-carbonyl, C₆₋₁₀ aryl-carbonyl, formyloxy, C₁₋₆ alkyl-carbonyloxy, C₆₋₁₀ aryl-carbonyloxy, carboxyl, C₁₋₆ alkoxycarbonyl, C₇₋₁₁ aralkyloxy-carbonyl, carbamoyl, an optionally halogenated C₁₋₄ alkyl, oxo, amidino, imino, amino, mono-C₁₋₄ alkylamino, di-C₁₋₄ alkylamino, 3- to 6-membered cyclic amino, C₁₋₃ alkylenedioxy, hydroxy, nitro, cyano, mercapto, sulfo, sulfino, phosphono, sulfamoyl, mono-C₁₋₆ alkylsulfamoyl, di-C₁₋₆ alkylsulfamoyl, C₁₋₆ alkylthio, C₆₋₁₀ arylthio, C₁₋₆ alkylsulfinyl, C₆₋₁₀ arylsulfinyl, C₁₋₆ alkylsulfonyl and C₆₋₁₀ arylsulfonyl;

R⁴ is (i) a hydrogen atom or (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino;

ring A is a 5- to 7-membered heterocyclic group optionally containing, besides carbon atoms and an oxygen atom, 1 to 3 hetero atoms selected from the group consisting of nitrogen, oxygen and sulfur, which may be substituted by 1 to 4 substituents selected from the group consisting of (i) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₁₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino, (ii) a halogen, (iii) C₁₋₆ alkoxy, (iv) C₆₋₁₀ aryloxy, (v) formyl, (vi) C₁₋₆ alkyl-carbonyl, (vii) C₆₋₁₀ aryl-carbonyl, (viii) formyloxy, (ix) C₁₋₆ alkyl-carbonyloxy, (x) C₆₋₁₀ aryl-carbonyloxy, (xi) carboxyl, (xii) C₁₋₆ alkoxy-carbonyl, (xiii) C₇₋₁₁ aralkyloxycarbonyl, (xiv) carbamoyl, (xv) an optionally halogenated C₁₋₄ alkyl, (xvi) oxo, (xvii) amidino, (xviii) imino, (xix) amino, (xx) mono-C₁₋₄ alkylamino, (xxi) di-C₁₋₄ alkylamino, (xxii) 3- to 6-membered cyclic amino, (xxiii) C₁₋₃ alkylenedioxy, (xxiv) hydroxy, (xxv) nitro, (xxvi) cyano, (xxvii) mercapto, (xxviii) sulfo, (xxix) sulfino, (xxx) phosphono, (xxxi) sulfamoyl, (xxxii) mono-C₁₋₆ alkylsulfamoyl, (xxxiii) di-C₁₋₆ alkylsulfamoyl, (xxxiv) C₁₋₆ alkylthio, (xxxv) C₆₋₁₀ arylthio, (xxxvi) C₁₋₆ alkylsulfinyl, (xxxvii) C₆₋₁₀ arylsulfinyl, (xxxviii) C₁₋₆ alkylsulfonyl and (xxxix) C₆₋₁₀ arylsulfonyl; and

ring B is a benzene ring which may be substituted by 1 or 2 substituents selected from the group consisting of (i) a halogen, (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino, (iii) an amino group which may be substituted by 1 or 2 substituents selected from the group consisting of a C₁₋₆ alkyl, C₂₋₆ alkenyl, C₂₋₆ alkynyl, C₃₋₆ cycloalkyl and C₆₋₁₄ aryl group, each of which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino, (iv) a C₁₋₆ alkanoylamino group, (v) a C₁₋₆ alkoxy group which may be substituted by 1 to 3 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino or (vi) a C₁₋₃ alkylenedioxy group.

(3) In another embodiment, the melatonin agonist is a compound of Formula III above, wherein the moiety of Formula I is:

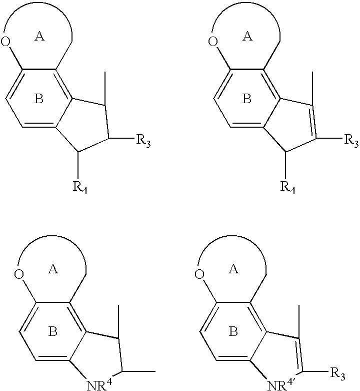

wherein R^{4′} is an optionally substituted hydrocarbon group and the other symbols are as defined above.

(4) In another embodiment, the melatonin agonist is a compound of the above (1), further having the structure of Formula IV:

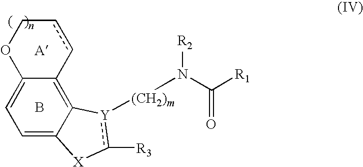

wherein ring A′ is an optionally substituted, oxygen-containing heterocyclic ring;

n is an integer of 0 to 2;

[symbol] are each independently a single bond or a double bond; and the other symbols are as defined above.

(5) In another embodiment, the melatonin agonist is as shown in above (1), wherein R¹ is (i) an optionally substituted C₁₋₆ alkyl group, (ii) an optionally substituted C₃₋₆ cycloalkyl group, (iii) an optionally substituted C₂₋₆ alkenyl group, (iv) an optionally substituted C₆₋₁₄ aryl group, (v) an optionally substituted mono- or di-C₁₋₆ alkylamino group, (vi) an optionally substituted C₆₋₁₄ arylamino group, or (vii) an optionally substituted 5- or 6-membered nitrogen-containing heterocyclic group.

(6) In another embodiment, the melatonin agonist is a compound of the above (1), wherein R¹ is an optionally halogenated C₁₋₆ alkyl group.

(7) In another embodiment, the melatonin agonist is a compound of the above (1), wherein R² is a hydrogen atom or an optionally substituted C₁₋₆ alkyl group.

(8) In another embodiment, the melatonin agonist is a compound of the above (1), wherein R² is a hydrogen atom.

(9) In another embodiment, the melatonin agonist is a compound of the above (1), wherein R³ is a hydrogen atom or an optionally substituted hydrocarbon group.

(10) In another embodiment, the melatonin agonist is a compound of the above (1), wherein R³ is a hydrogen atom.

(11) In another embodiment, the melatonin agonist is a compound of the above (1), wherein R⁴ is a hydrogen atom or an optionally substituted C₁₋₆ alkyl group.

(12) In another embodiment, the melatonin agonist is a compound of the above (1), wherein X is CHR⁴.

(13) In another embodiment, the melatonin agonist is a compound of the

above (1), wherein X is CHR⁴ and [symbol] is a single bond.

(14) In another embodiment, the melatonin agonist is a compound of the above (13), wherein X is CH₂.

(15) In another embodiment, the melatonin agonist is a compound of the above (1), wherein X is NR⁴.

(16) In another embodiment, the melatonin agonist is a compound of the above (1), wherein Y is C or CH.

(17) In another embodiment, the melatonin agonist is a compound of the above (1), wherein Y is CH.

(18) In another embodiment, the melatonin agonist is a compound of the above (1), wherein m is 2.

(19) In another embodiment, the melatonin agonist is a compound of the above (1), wherein ring A is a tetrahydrofuran ring.

(20) In another embodiment, the melatonin agonist is a compound of the above (1), wherein ring A is unsubstituted.

(21) In another embodiment, the melatonin agonist is a compound of the above (1), wherein ring B is unsubstituted.

(22) In another embodiment, the melatonin agonist is a compound of the above (4), wherein n is 0 or 1.

(23) In another embodiment, the melatonin agonist is a compound of the above (1) which is a compound of the Formula V:

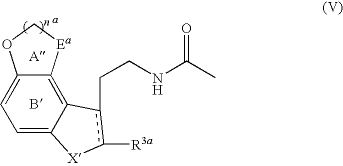

wherein R^{1b} is C₁₋₆ alkyl;

X′ is CH₂, NH or NCHO; —

[symbol] is a single bond or double bond;

R^{3a} is a hydrogen atom or a phenyl;

E^{a} is CH₂CH₂, CH═CH, CH₂O, CH═N, CONH or CH₂NH;

n^{a} is 0 or 1;

ring A″ is a 5- or 6-membered oxygen-containing heterocyclic ring which may be substituted by 1 or 2 C₁₋₆ alkyl optionally substituted by a hydroxyl; and ring B′ is a benzene ring which may be substituted by a halogen.

(24) In another embodiment, the melatonin agonist is a compound of the above (23), wherein [symbol] is single bond and X′ is NH.

(25) In another embodiment, the melatonin agonist is a compound of the above (1), which is (S)—N-[2-(1,6,7,8-tetrahydro-2H-indeno[5,4-b]furan-8-yl)ethyl]propionamide.

(26) In another embodiment, the melatonin agonist is a compound of the above (1), which is N-[2-(1,6,7,8-tetrahydro-2H-furo[3,2-e]indol-8-yl)ethyl]propionamide.

(27) In another embodiment, the melatonin agonist is a compound of the above (1), which is N-[2-(1,6,7,8-tetrahydro-2H-furo[3,2-e]indol-8-yl)ethyl]butyramide.

(28) In another embodiment, the melatonin agonist is a compound of the above (1), which is N-[2-(7-phenyl-1,6-dihydro-2H-indeno[5,4-b]furan-8-yl)ethyl]propionamide.

(29) In another embodiment, the melatonin agonist is a compound of the above (1), which is N-[2-(7-phenyl-1,6-dihydro-2H-indeno[5,4-b]furan-8-yl)ethyl]butyramide.

(30) In another embodiment, the melatonin agonist is a compound of Formula VI:

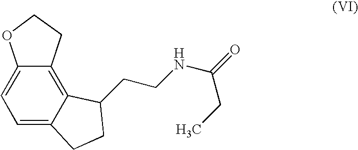

(31) In another embodiment, the melatonin agonist is a compound of Formula VII:

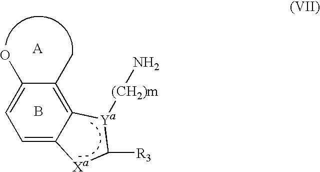

wherein X^{a} represents CHR^{4a}, NR^{4a}, O or S in which R^{4a} represents a hydrogen atom or an optionally substituted hydrocarbon group; Y^{a} represents C, CH or N, provided that when X^{a} is NH, Y^{a} is CH or N; and the other symbols are as defined above, or a salt thereof.

In another embodiment, the melatonin agonist is a compound of any of (1)-(31) above, or a pharmaceutically acceptable salt thereof.

The “hydrocarbon group” in an “optionally substituted hydrocarbon group” as referred to herein includes, for example, an aliphatic hydrocarbon group, a mono-cyclic saturated hydrocarbon group, an aromatic hydrocarbon group, etc., and this optionally has from 1 to 16 carbon atoms. Illustrative examples include an alkyl group, an alkenyl group, an alkynyl group, a cycloalkyl group, an aryl group, etc.

The “alkyl group” is, for example, a lower alkyl group and generally includes C₁₋₆ alkyl groups such as methyl, ethyl, propyl, isopropyl, butyl, isobutyl, sec-butyl, tert-butyl, pentyl, hexyl, etc.

The “alkenyl group” is, for example, a lower alkenyl group and generally includes C₂₋₆ alkenyl groups such as vinyl, 1-propenyl, allyl, isopropenyl, butenyl, isobutenyl, etc.

The “alkynyl group” is, for example, a lower alkynyl group and generally includes C₂₋₆ alkynyl groups such as ethynyl, propargyl, 1-propynyl, etc.

The “cycloalkyl group” is, for example, a lower cycloalkyl group and generally includes C₃₋₆ cycloalkyl groups such as cyclopropyl, cyclobutyl, cyclopentyl, cyclohexyl, etc.

The “aryl group” is illustratively a C₆₋₁₄ aryl group, including, for example, phenyl, 1-naphthyl, 2-naphthyl, biphenylyl, 2-anthryl, etc.

The substituents for the “hydrocarbon group” of the “optionally substituted hydrocarbon group” include, for example, a halogen atom (e.g., fluorine, chlorine, bromine, iodine, etc.), a nitro group, a cyano group, a hydroxy group, an optionally halogenated lower alkyl group (e.g., an optionally halogenated C₁₋₆ alkyl group such as methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, isobutyl, sec-butyl, tert-butyl, 4,4,4-trifluorobutyl, pentyl, isopentyl, neopentyl, 5,5,5-trifluoropentyl, hexyl, 6,6,6-trifluorohexyl, etc.), a lower alkoxy group (e.g., a C₁₋₆ alkoxy group such as methoxy, ethoxy, propoxy, isopropoxy, butoxy, isobutoxy, pentyloxy, hexyloxy, etc.), an amino group, a mono-lower alkylamino group (e.g., a mono-C₁₋₆ alkylamino group such as methylamino, ethylamino, etc.), a di-lower alkylamino group (e.g., a di-C₁₋₆ lower alkylamino group such as dimethylamino, diethylamino, etc.), a carboxyl group, a lower alkylcarbonyl group (e.g., a C₁₋₆ alkyl-carbonyl group such as acetyl, propionyl, etc.), a lower alkoxycarbonyl group (e.g., a C₁₋₆ alkoxy-carbonyl group such as methoxycarbonyl, ethoxycarbonyl, propoxycarbonyl, butoxycarbonyl, etc.), a carbamoyl group, a mono-lower alkylcarbamoyl group (e.g., a mono-C₁₋₆ alkyl-carbamoyl group such as methylcarbamoyl, ethylcarbamoyl, etc.), a di-lower alkylcarbamoyl group (e.g., a alkyl-carbamoyl group such as dimethylcarbamoyl, diethylcarbamoyl, etc.), an arylcarbamoyl group (e.g., a C₆₋₁₀ aryl-carbamoyl group such as phenylcarbamoyl, naphthylcarbamoyl, etc.), an aryl group (e.g., a C₆₋₁₀ aryl group such as phenyl, naphthyl, etc.), an aryloxy group (e.g., a C₆₋₁₀ aryloxy group such as phenyloxy, naphthyloxy, etc.), an optionally halogenated lower alkylcarbonylamino group (e.g., an optionally halogenated C₁₋₆ alkylcarbonylamino group such as acetylamino, trifluoroacetylamino, etc.), an oxo group, etc. The “hydrocarbon group” of the “optionally substituted hydrocarbon group” may have 1 to 5 or 1 to 3 substituents selected from those mentioned above, at any substitutable positions in the group. When the number of the substituents is two or more, each of the substituents may be the same or different.

The “heterocyclic group” in “optionally substituted heterocyclic group” as referred to herein includes, for example, a 5- to 14-membered (or 5- to 10-membered), mono- to tri-cyclic (e.g. mono- or di-cyclic) heterocyclic group, each having 1 or 2, 1-3 or 1 to 4, kinds of hetero atoms selected from nitrogen, oxygen and sulfur, in addition to carbon atoms. Concretely, it includes, for example, a 5-membered heterocyclic group having 1 to 4 hetero atoms selected from oxygen, sulfur and nitrogen, in addition to carbon atoms, such as 2- or 3-thienyl, 2- or 3-furyl, 1, 2- or 3-pyrrolyl, 1-, 2- or 3-pyrrolidinyl, 2-, 4- or 5-oxazolyl, 3-, 4- or 5-isoxazolyl, 2-, 4- or 5-thiazolyl, 3-, 4- or 5-isothiazolyl, 3-, 4- or 5-pyrazolyl, 2-, 3- or 4-pyrazolidinyl, 2-, 4-, or 5-imidazolyl, 1,2,3-triazolyl, 1,2,4-triazolyl, 1H- or 2H-tetrazolyl; a 6-membered heterocyclic group having 1 to 4 hetero atoms selected from oxygen, sulfur and nitrogen atoms, in addition to carbon atoms, such as 2-, 3- or 4-pyridyl, N-oxido-2-, 3- or 4-pyridyl, 2-, 4- or 5-pyrimidinyl, N-oxido-2-, 4- or 5-pyrimidinyl, thiomorpholinyl, morpholinyl, piperidino, 2-, 3- or 4-piperidyl, thiopyranyl, 1,4-oxazinyl, 1,4-thiazinyl, 1,3-thiazinyl, piperazinyl, triazinyl, 3- or 4-pyridazinyl, pyrazinyl, N-oxido-3- or 4-pyridazinyl; a di- or tricyclic condensed heterocyclic group having 1 to 4 hetero atoms selected from oxygen, sulfur and nitrogen atoms, in addition to carbon atoms (illustratively a group to be formed by condensing the above-mentioned 5- or 6-membered cyclic group with one or two 5- or 6-membered cyclic groups each optionally having 1 to 4 hetero atoms selected from oxygen, sulfur and nitrogen atoms, in addition to carbon atoms), such as indolyl, benzofuryl, benzothiazolyl, benzoxazolyl, benzimidazolyl, quinolyl, isoquinolyl, phthalazinyl, quinazolinyl, quinoxalinyl, indolidinyl, quinolidinyl, 1,8-naphthyridinyl, dibenzofuranyl, carbazolyl, acridinyl, phenanthridinyl, chromanyl, phenothiazinyl, phenoxazinyl, etc. Of these, preferred are 5- to 7-membered (e.g. a 5- or 6-membered) heterocyclic groups each having 1 to 3 hetero atoms selected from oxygen, sulfur and nitrogen atoms, in addition to carbon atoms.

The substituents for the “heterocyclic group” of the “optionally substituted heterocyclic group” include, for example, a halogen atom (e.g., fluorine, chlorine, bromine, iodine, etc.), a lower alkyl group (e.g., a C₁₋₆ alkyl group such as methyl, ethyl, propyl, isopropyl, butyl, isobutyl, sec-butyl, tert-butyl, pentyl, hexyl, etc.), a cycloalkyl group (e.g., a C₃₋₆ cycloalkyl group such as cyclopropyl, cyclobutyl, cyclopentyl, cyclohexyl, etc.), a lower alkynyl group (e.g., a C₂₋₆ alkynyl group such as ethynyl, 1-propynyl, propargyl, etc.), a lower alkenyl group (e.g., a C₂₋₆ alkenyl group such as vinyl, allyl, isopropenyl, butenyl, isobutenyl, etc.), an aralkyl group (e.g., a C₇₋₁₁ aralkyl group such as benzyl, .alpha.-methylbenzyl, phenethyl, etc.), an aryl group (e.g., a C₆₋₁₀ aryl group such as phenyl, naphthyl, etc.), a lower alkoxy group (e.g., a C₁₋₆ alkoxy group such as methoxy, ethoxy, propoxy, isopropoxy, butoxy, isobutoxy, sec-butoxy, tert-butoxy, etc.), an aryloxy group (e.g., a C₆₋₁₀ aryloxy group such as phenoxy, etc.), a lower alkanoyl group (e.g., formyl, a C₁₋₆ alkyl-carbonyl group such as acetyl, propionyl, butyryl, isobutyryl, etc.), an arylcarbonyl group (e.g., a C₆₋₁₀ aryl-carbonyl group such as benzoyl, naphthoyl, etc.), a lower alkanoyloxy group (e.g., formyloxy, a C₁₋₆ alkyl-carbonyloxy group such as acetyloxy, propionyloxy, butyryloxy, isobutyryloxy, etc.), an arylcarbonyloxy group (e.g., a C₆₋₁₀ aryl-carbonyloxy group such as benzoyloxy, naphthoyloxy, etc.), a carboxyl group, a lower alkoxycarbonyl group (e.g., a C₁₋₆ alkoxy-carbonyl group such as methoxycarbonyl, ethoxycarbonyl, propoxycarbonyl, isopropoxycarbonyl, butoxycarbonyl, isobutoxycarbonyl, tert-butoxycarbonyl, etc.), an aralkyloxycarbonyl group (e.g., a C₇₋₁₁ aralkyloxycarbonyl group such as benzyloxycarbonyl, etc.), a carbamoyl group, a mono-, di- or tri-halogeno-lower alkyl group (e.g., a mono-, di- or tri-halogeno-C₁₋₄ alkyl group such as chloromethyl, dichloromethyl, trifluoromethyl, 2,2,2-trifluoroethyl, etc.), an oxo group, an amidino group, an imino group, an amino group, a mono-lower alkylamino group (e.g., a mono-C₁₋₄ alkylamino group, such as methylamino, ethylamino, propylamino, isopropylamino, butylamino, etc.), a di-lower alkylamino group (e.g., a di-C₁₋₄ alkylamino group such as dimethylamino, diethylamino, dipropylamino, diisopropyl amino, dibutylamino, methylethylamino, etc.), a 3- to 6-membered cyclic amino group optionally having 1 to 3 hetero atoms selected from oxygen, sulfur and nitrogen atoms, in addition to carbon atoms and one nitrogen atom (e.g., a 3- to 6-membered cyclic amino group such as aziridinyl, azetidinyl, pyrrolidinyl, pyrrolinyl, pyrrolyl, imidazolyl, pyrazolyl, imidazolidinyl, piperidyl, morpholinyl, dihydropyridyl, pyridyl, N-methylpiperazinyl, Nethylpiperazinyl, etc.), an alkylenedioxy group (e.g., a C₁₋₃ alkylenedioxy group such as methylenedioxy, ethylenedioxy, etc.), a hydroxy group, a nitro group, a cyano group, a mercapto group, a sulfo group, a sulfino group, a phosphono group, a sulfamoyl group, a monoalkylsulfamoyl group (e.g., a mono-C₁₋₆ alkylsulfamoyl group such as N-methylsulfamoyl, N-ethylsulfamoyl, N-propylsulfamoyl, Nisopropylsulfamoyl, N-butylsulfamoyl, etc.), a dialkylsulfamoyl group (e.g., a di-C₁₋₆ alkylsulfamoyl group such as N,N-dimethylsulfamoyl, N,N-diethylsulfamoyl, N,N-dipropylsulfamoyl, N,N-dibutylsulfamoyl, etc.), an alkylthio group (e.g., C₁₋₆ alkylthio group such as methylthio, ethylthio, propylthio, isopropylthio, butylthio, sec-butylthio, tert-butylthio, etc.), an arylthio group (e.g., a C₆₋₁₀ arylthio group such as phenylthio, naphthylthio, etc.), a lower alkylsulfinyl group (e.g., a C₁₋₆ alkylsulfinyl group such as methylsulfinyl, ethylsulfinyl, propylsulfinyl, butylsulfinyl, etc.), an arylsulfinyl group (e.g., a C₆₋₁₀ arylsulfinyl group such as phenylsulfinyl, naphthylsulfinyl, etc.), a lower alkylsulfonyl group (e.g., a C₁₋₆ alkylsulfonyl group such as methylsulfonyl, ethylsulfonyl, propylsulfonyl, butylsulfonyl, etc.), an arylsulfonyl group (e.g., a C₆₋₁₀ arylsulfonyl group such as phenylsulfonyl, naphthylsulfonyl, etc.), etc.

The “heterocyclic group” of the “optionally substituted heterocyclic group” may have 1 to 5 or 1 to 3 substituents selected from those mentioned above, at any substitutable positions in the group. In the case that the group has two or more substituents, these substituents may be the same or different.

The “optionally substituted amino group” as referred to herein includes amino groups each optionally having one or two substituents of, for example, the above-mentioned “optionally substituted hydrocarbon groups”. Illustrative substituents for the above “amino group” include, for example, an optionally substituted C₁₋₆ alkyl group and an optionally substituted C₆₋₁₀ aryl group. The substituents which the “C₁₋₆ alkyl group” or the “C₆₋₁₀ aryl group” may optionally have are, for example, the same ones as the above-mentioned “hydrocarbon group” may optionally have.

The “lower alkyl group” for “optionally substituted lower alkyl group” as referred to herein includes, for example, a C₁₋₆ alkyl group such as methyl, ethyl, propyl, isopropyl, butyl, isobutyl, sec-butyl and tert-butyl. The lower alkyl group may optionally have 1 to 3 substituents, such as the same ones as the above-mentioned “hydrocarbon group” may optionally have.

The “lower alkoxy group” in “optionally substituted lower alkoxy group” as referred to herein includes, for example, a C₁₋₆ alkoxy group such as methoxy, ethoxy, propoxy, isopropoxy, butoxy, isobutoxy, sec-butoxy and tertbutoxy. The lower alkoxy group may optionally have 1 to 3 substituents, such as the same ones as the above-mentioned “hydrocarbon group” may optionally have.

The “optionally substituted benzene ring” as referred to herein includes, for example, a benzene ring which may optionally have one or two substituents selected from, a halogen atom (e.g., fluorine, chlorine, bromine, iodine, etc.), an optionally substituted hydrocarbon group, an optionally substituted amino group, an amido group (e.g., a C₁₋₃ acyl amino group such as formamido, acetamido, etc.), an optionally substituted lower alkoxy group and a lower alkylenedioxy group (e.g., a C₁₋₃ alkylenedioxy group such as methylenedioxy, ethylenedioxy, etc.), at any substitutable positions in the ring.

For these “optionally substituted hydrocarbon group”, “optionally substituted amino group” and “optionally substituted lower alkoxy group”, the same ones as those described in detail hereinabove are referred to. In the case that these “hydrocarbon group”, “amino group” and “lower alkoxy group” each have two or more substituents, these substituents may be the same or different.

The “optionally substituted benzene ring” is optionally a benzene ring optionally substituted by 1 or 2 substituents selected from a halogen atom (e.g., fluorine, chlorine, etc.), a C₁₋₆ alkyl group (e.g., methyl, ethyl, etc.) and a mono-C₁₋₆ alkylamino group.

In the above-mentioned formulae, R¹ represents an optionally substituted hydrocarbon group, an optionally substituted amino group or an optionally substituted heterocyclic group.

The “hydrocarbon group” of the “optionally substituted hydrocarbon group” represented by R′ is, for example, an alkyl group (e.g., a C₁₋₆ alkyl group such as methyl, ethyl, propyl, isopropyl, etc.), an alkenyl group (e.g., C₂₋₆ alkenyl group such as vinyl, etc.), an alkynyl group (e.g., a C₂₋₆ alkynyl group such as ethynyl), a cycloalkyl group (e.g., a C₃₋₆ cycloalkyl group such as cyclopropyl, cyclobutyl, cyclopentyl, cyclohexyl, etc.), or an aryl group (e.g., a C₆₋₁₄ aryl group such as phenyl, etc.), or an alkyl group (e.g., a C₁₋₆ alkyl group such as methyl, etc.) or a cycloalkyl group (e.g., a C₃₋₆ cycloalkyl group such as cyclopropyl, etc.). These “alkyl group”, “alkenyl group”, “alkynyl group”, “cycloalkyl group” and “aryl group” each may have 1 to 5 or 1 to 3 substituents, such as the same ones as the above-mentioned “hydrocarbon group” may optionally have halogen atoms such as fluorines.

Illustrative substituents for the “optionally substituted amino group” represented by R¹, are one or two substituents selected from, for example, an optionally substituted lower alkyl group and an optionally substituted aryl group. The “lower alkyl group” includes, for example, a C₁₋₆ alkyl group such as methyl, ethyl, propyl, isopropyl, butyl, isobutyl, sec-butyl and tert-butyl. The “lower alkyl group” may optionally have 1 to 3 substituents, such as the same ones as the above-mentioned “hydrocarbon group” may optionally have. The “aryl group” includes, for example, a C₆₋₁₀ aryl group such as phenyl, etc. The “aryl group” may optionally have 1 to 5 or 1 to 3 substituents, such as the same ones as the above-mentioned “hydrocarbon group” may optionally have, for example, a halogen atom such as fluorine and chlorine and a C₁₋₆ alkoxy group such as methoxy and ethoxy. The “optionally substituted amino group” includes, for example, a phenylamino group substituted by, 1 to 3 lower alkoxy groups (e.g., C₁₋₄ alkoxy groups such as methoxy, etc.) or a monoalkylamino group substituted by one lower alkyl group (e.g., a C₁₋₄ alkyl group such as methyl, ethyl, propyl, butyl, tert-butyl, etc.).

The “heterocyclic group” of the “optionally substituted heterocyclic group” represented by R¹ is, for example, a 5- or 6-membered heterocyclic group having 1 to 3 hetero atoms selected from nitrogen, oxygen and sulfur atoms in addition to carbon atoms. Illustratively, it includes 1-, 2- or 3-pyrrolidinyl, 2- or 4-imidazolinyl, 2-, 3- or 4-pyrazolidinyl, piperidino, 2-, 3- or 4-piperidyl, 1- or 2 piperazinyl, morpholinyl, 2- or 3-thienyl, 2-, 3- or 4-pyridyl, 2- or 3-furyl, pyrazinyl, 2-pyrimidinyl, 3-pyrrolyl, 3-pyridazinyl, 3-isothiazolyl and 3-isoxazolyl, or 6-membered nitrogen-containing heterocyclic group (e.g., pyridyl, etc.).

Illustrative substituents for the “optionally substituted heterocyclic group” represented by R¹ include, for example, a halogen atom (e.g., chlorine, fluorine, etc.), a C₁₋₆ alkyl group (e.g., methyl, ethyl, etc.), a C₁₋₆ alkoxy group (e.g., methoxy, ethoxy, etc.) and an aralkyloxycarbonyl group (e.g., a C₇₋₁₂ aralkyloxy-carbonyl group such as benzyloxycarbonyl, etc.).

R¹ is, for example, (i) an optionally substituted lower alkyl group, (ii) an optionally substituted lower cycloalkyl group, (iii) an optionally substituted lower alkenyl group, (iv) an optionally substituted aryl group, (v) an optionally substituted mono- or di-lower alkylamino group, (vi) an optionally substituted arylamino group or (vii) an optionally substituted 5- or 6-membered nitrogen-containing heterocyclic group.

The “lower alkyl group” is optionally a C₁₋₆ alkyl group such as methyl, ethyl, propyl, isopropyl, butyl, pentyl and hexyl. The “lower cycloalkyl group” is optionally a C₃₋₆ cycloalkyl group such as cyclopropyl, cyclobutyl, cyclopentyl and cyclohexyl. The “lower alkenyl group” is optionally a C₂₋₆ alkenyl group such as vinyl, 1-propenyl and butenyl. The “aryl group” is optionally a C₆₋₁₀ aryl group such as phenyl, 1-naphthyl and 2-naphthyl. The “lower alkylamino group” is optionally a mono- or di-C₁₋₆ alkylamino group such as methylamino, ethylamino, propylamino, isopropylamino, butyl amino, tert-butylamino, dimethylamino, diethylamino and methylethylamino. The “arylamino group” is optionally a C₆₋₁₀ arylamino group such as phenylamino. The “5- or 6-membered nitrogen-containing heterocyclic group” is, for example, optionally 2-, 3- or 4-pyridyl or the like. These groups may each optionally have 1 to 5 substituents such as those referred to the mentioned-above “hydrocarbon group” may optionally have.

Alternatively, R¹ is (i) a C₁₋₆ alkyl group optionally substituted by 1 to 4 substituents selected from a halogen atom and a C₁₋₆ alkoxy group, (ii) a C₃₋₆ cycloalkyl group, (iii) a C₂₋₆ alkenyl group, (iv) a C₆₋₁₀ aryl group optionally substituted by 1 to 4 substituents selected from a C₁₋₆ alkoxy group, a nitro group, a halogeno-C₁₋₆ alkyl-carbonylamino group and a halogen atom, (v) a mono- or di-C₁₋₆ alkylamino group, (vi) a C₆₋₁₀ arylamino group optionally substituted by one to three C₁₋₆ alkoxy groups, or (vii) a 6-membered nitrogen-containing heterocyclic group optionally substituted by one or two C₇₋₁₁ aralkyloxycarbonyl groups. Alternatively, R¹ is an optionally halogenated C₁₋₆ alkyl group (e.g., methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, isobutyl, sec-butyl, tert-butyl, 4,4,4-trifluorobutyl, pentyl, isopentyl, neopentyl, 5,5,5-trifluoropentyl, hexyl, 6,6,6-trifluorohexyl, etc.), a C₃₋₆ cycloalkyl group (e.g., cyclopropyl, cyclobutyl, cyclopentyl, cyclohexyl, etc.) or a mono-C₁₋₆ alkylamino group (e.g., methylamino, ethylamino, propylamino, isopropylamino, butylamino, tert-butylamino, etc.) Among others, R¹ is optionally a halogenated C₁₋₆ alkyl group or a mono-C₁₋₆ alkylamino group, especially an optionally halogenated C₁₋₆ alkyl, in particular C₁₋₃ alkyl group (e.g., methyl, ethyl, propyl, etc.).

In the above-mentioned formulae, R² represents a hydrogen atom or an optionally substituted hydrocarbon group. In one embodiment, R² is a hydrogen atom, an optionally substituted lower (C₁₋₆) alkyl group, or a hydrogen atom.

In the above-mentioned formulae, R³ represents a hydrogen atom, an optionally substituted hydrocarbon group or optionally substituted heterocyclic group.

The “hydrocarbon group” of the “optionally substituted hydrocarbon group” represented by R³ is optionally, for example, an alkyl group (e.g., a C₁₋₆ alkyl group such as methyl, ethyl, propyl, isopropyl, etc.), an alkenyl group (e.g., a C₂₋₆ alkenyl group such as vinyl, etc.), an alkynyl group (e.g., a C₂₋₆ alkynyl group such as ethynyl, etc.), a cycloalkyl group (e.g., a C₃₋₆ cycloalkyl group such as cyclopropyl, cyclobutyl, cyclopentyl, cyclohexyl, etc.) or an aryl group (e.g., a C₆₋₁₄ aryl group such as phenyl, etc.). Optionally, it is an alkyl group (e.g., a C₁₋₆ alkyl group such as methyl, etc.) or an aryl group (e.g., a C₆₋₁₄ aryl groups such as phenyl, etc.). These “alkyl group”, “alkenyl group”, “alkynyl group”, “cycloalkyl group” and “aryl group” each may optionally have 1 to 5 or 1 to 3 substituents such as the same ones the mentioned-above “hydrocarbon group” may optionally have (e.g., halogen atoms such as fluorines, etc.).

The “heterocyclic group” of the “optionally substituted heterocyclic group” represented by R³ is optionally a 5- or 6-membered heterocyclic group having 1 to 3 hetero atoms selected from nitrogen, oxygen and sulfur atoms, in addition to carbon atoms. Illustratively, it includes, for example, 1-, 2- or 3-pyrrolidinyl, 2- or 4-imidazolinyl, 2-, 3- or 4-pyrazolidinyl, piperidino, 2-, 3- or 4-piperidyl, 1- or 2-piperazinyl, morpholinyl, 2- or 3-thienyl, 2-, 3- or 4-pyridyl, 2- or 3-furyl, pyrazinyl, 2-pyrimidinyl, 3-pyrrolyl, 3-pyridazinyl, 3-isothiazolyl, 3-isoxazolyl, etc or a 6-membered nitrogen-containing heterocyclic group (e.g., pyridyl, etc.).

Preferred substituents for the “optionally substituted heterocyclic group” represented by R³ include, for example, a halogen atom (e.g., chlorine, fluorine, etc.), a C₁₋₆ alkyl group (e.g., methyl, ethyl, etc.), a C₁₋₆ alkoxy group (e.g., methoxy, ethoxy, etc.), an aralkyloxycarbonyl group (e.g., a C₇₋₁₂ aralkyloxycarbonyl group such as benzyloxycarbonyl, etc.), an amino group, a mono-C₁₋₆ alkylamino group (e.g., methylamino, ethylamino, etc.) a di-C₁₋₆ alkylamino group (e.g., dimethylamino, diethylamino, etc.) etc.

R³ is, for example a hydrogen atom, an optionally substituted lower alkyl group, an optionally substituted aryl group, an optionally substituted 5- or 6-membered heterocyclic group, a lower alkyl group, an optionally substituted C₆₋₁₀ aryl group, or an optionally substituted 6-membered nitrogen-containing heterocyclic group.

The above substituents include, for example, a hydrogen atom, a C₁₋₆ alkyl group, a C₁₋₆ alkoxy group, an amino group, a mono-C₁₋₆ alkylamino group, a di-C₁₋₆ alkylamino group, etc.

In one embodiment, R³ is, for example, a hydrogen atom, a phenyl group or a 2-, 3- or 4-pyridyl group.

In the above-mentioned formulae, X represents CHR⁴, NR⁴, O or S in which R⁴ represents a hydrogen atom or an optionally substituted hydrocarbon group.

X^{a} represents CHR^{4a}, NR^{4a}, O or S in which R^{4a} represents a hydrogen atom or an optionally substituted hydrocarbon group.

R⁴ and R^{4a} are optionally a hydrogen atom or an optionally substituted lower (C₁₋₆) alkyl group, respectively.

X^{a} is optionally CHR^{4a} in which R⁴ is as defined above, O or S. Or, X is CHR⁴ or NR⁴ in which R⁴ is as defined above.

X^{a} is optionally CHR^{4a} or NR^{4a} in which R^{4a} is as defined above.

In the above formulae, Y represents C, CH or N. Y^{a} represents C, CH or N.

In the above-mentioned formulae, ring A or ring A′ represents an optionally substituted, 5- to 7-membered oxygen-containing heterocyclic ring.

The “5- to 7-membered oxygen-containing heterocyclic ring” includes 5- to 7-membered (e.g. 5- or 6-membered) heterocyclic rings optionally having 1 to 3 hetero atoms selected from nitrogen, oxygen and sulfur atoms, in addition to carbon atoms and an oxygen atom.

Illustrative examples of suitable melatonin agonists include:
N-[2-(1,6,7,8-tetrahydro-2H-indeno[5,4-b[furan-8-yl)ethyl]acetamide
N-[2-(1,6,7,8-tetrahydro-2H-indeno[5,4-b]furan-8-yl)ethyl)]butyramide,
N-[2-(1,6,7,8-tetrahydro-2H-indeno[5,4-b]furan-8-yl)ethyl]propionamide,
N-[2-(3,7,8,9-tetrahydropyrano[3,2-e]indol-1-yl)ethyl]propionamide,
N-[2-(3,7,8,9-tetrahydropyrano[3,2-e]indol-1-yl)]butyramide,
N-[2-(1,2,3,7,8,9-hexahydropyrano[3,2-e]indol-1-yl)ethyl]propionamide,
N-[2-(1,2,3,7,8,9-hexahydropyrano[3,2-e]indol-1-yl)ethyl]butyramide,
N-[2-(4-fluoro-1,6,7,8-tetrahydro-2H-indeno[5,4-b]furan-8-yl)ethyl]butyramide,
N-[2-(4-fluoro-1,6,7,8-tetrahydro-2H-indeno[5,4-b]furan-8-yl)ethyl]propionamide,
N-[2-(5-fluoro-3,7,8,9-tetrahydrocyclopenta[f][1]benzopyran-9-yl)ethyl]propionamide,
(S)—N-[2-(1,6,7,8-tetrahydro-2H-indeno[5,4-b]furan-8-yl)ethyl]propionamide,
(R)—N-[2-(1,6,7,8-tetrahydro-2H-indeno[5,4-b]furan-8-yl)ethyl]propionamide,
N-[2-(1,6,7,8-tetrahydro-2H-indeno[5,4-b]furan-8-yl)ethyl]butyramide,
N-[2-(1,6-dihydro-2H-indeno[5,4-b]furan-8-yl)ethyl]acetamide,
N-[2-(1,6-dihydro-2H-indeno[5,4-b]furan-8-yl)ethyl]propionamide,
N-[2-(1,6-dihydro-2H-indeno[5,4-b]furan-8-yl)ethyl]butyramide,
N-[2-(7,8-dihydro-6H-indeno[4,5-d]-1,3-dioxol-8-yl)ethyl]propionamide,
N-[2-(7,8-dihydro-6H-indeno[4,5-d]-1,3-dioxol-8-yl)ethyl]butyramide,
N-[2-(2,3,8,9-tetrahydro-7H-indeno[4,5-b]-1,4-dioxyn-9-yl)ethyl]propionamide,
N-[2-(2,3,8,9-tetrahydro-7H-indeno[4,5-b]-1,4-dioxyn-9-yl)ethyl]butyramide,
N-[2-(1,6,7,8-tetrahydro-2H-furo[3,2-e]indol-8-yl)ethyl]propionamide,
N-[2-(1,6,7,8-tetrahydro-2H-furo[3,2-e]indol-8-yl)ethyl]butyramide,
N-[2-(7-phenyl-1,6-dihydro-2H-indeno[5,4-b]furan-8-yl)ethyl]propionamide,
N-[2-(7-phenyl-1,6-dihydro-2H-indeno[5,4-b]furan-8-yl)ethyl]butyramide,
N-[2-(1,6,7,8-tetrahydro-2H-indeno[5,4-b]furan-8-yl)ethyl]acetamide,
N-[2-(1,6,7,8-tetrahydro-2H-indeno[5,4-b]furan-8-yl)ethyl]propionamide,
N-[2-(5-fluoro-3,7,8,9-tetrahydrocyclopenta[f][1]-benzopyran-9-yl)ethyl]propionamide,
N-[2-(5-fluoro-1,2,3,7,8,9-hexahydrocyclopenta[f][1]benzopyran-9-yl)ethyl]propionamide, or pharmaceutically acceptable salts of any of the foregoing.

The foregoing compounds can be prepared in any suitable manner, for example according to U.S. Pat. No. 6,034,239, the entirety of which is hereby incorporated herein by reference.

Suitable melatonin agonists may be in free form or in pharmaceutically acceptable salt or complex form. “Pharmaceutically acceptable salts,” or “salts,” include the salt of an melatonin agonist prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, beta-hydroxybutyric, galactaric and galacturonic acids.

In one embodiment, acid addition salts are prepared from the free base forms using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. Non-limiting examples of pharmaceutically acceptable salts of melatonin agonists include those salt-forming acids and bases that do not substantially increase the toxicity of the compound. Non-limiting examples of suitable salts include salts of alkali metals such as magnesium, potassium and ammonium, salts of mineral acids such as hydrochloric, hydriodic, hydrobromic, phosphoric, metaphosphoric, nitric and sulfuric acids, as well as salts of organic acids such as tartaric, acetic, citric, malic, benzoic, glycollic, gluconic, gulonic, succinic, arylsulfonic, e.g. p-toluenesulfonic acids, and the like.

In other embodiments, an acid addition salt is reconverted to the free base by treatment with a suitable base. In a further embodiment, suitable acid addition salts of the melatonin agonist are halide salts, which are prepared using hydrochloric or hydrobromic acids. In still other embodiments, the basic salts are alkali metal salts, e.g., sodium salt.

Compositions of the invention can comprise one or more melatonin agonists in any suitable amount. In one embodiment, a composition of the invention comprises a melatonin agonist in an amount of about 1 μg to about 1000 mg, about 1 μg to about 500 mg, about 1 μg to about 250 mg or about 1 μg to about 100 mg. Compositions of the invention typically comprise one or more melatonin agonists in a concentration of about 0.1 mg/ml to about 300 mg/ml, about 0.5 mg/ml to about 250 mg/ml, about 0.75 mg/ml to about 200 mg/ml, or about 1 mg/ml to about 100 mg/ml.

### Liquid Nasal Carrier

Compositions of the present invention comprise a liquid nasal carrier. As used herein, the phrase “liquid nasal carrier” or “liquid carrier” refers to a liquid vehicle (e.g. solution, emulsion, or suspension) designed for delivery of a drug to the nasal mucosa of a subject. The liquid nasal carrier can include one or more excipients such as diluents, solvents and/or co-solvents suitable for application to the nasal mucosa. Suitable diluents include aqueous or non-aqueous diluents or combination thereof. Examples of aqueous diluents include, but are not limited to, saline, water, water for injection (WFI), dextrose or combinations thereof.

In one embodiment, the liquid nasal carrier comprises a solvent such as a water miscible solvent. Non-limiting examples of suitable solvents include propylene glycol, alcohol, glycerol, isopropylalcohol and polyethylene glycol.

Any desired aqueous and/or non-aqueous diluents, solvents or co-solvents can be added in various concentrations and combinations to form a liquid nasal carrier in compositions of the invention. The liquid nasal carrier can be present in any suitable amount, for example about 10% to about 99%, about 20% to about 98%, about 30% to about 97%, by weight of the composition. In another embodiment, the liquid nasal carrier can be added to the other components of the composition in an amount sufficient to q.s. the composition to a desired final volume. In one embodiment, at least a portion of, at least about 20% of, at least about 30%, at least about 40%, at least about 50%, at least about 60%, at least about 70%, at least about 80%, or at least about 90%, by weight, of the melatonin agonist/antagonist is in dissolved and/or solubilized form in the liquid nasal carrier.

### Pharmaceutical Excipients

Compositions of the invention optionally comprise one or more additional pharmaceutically acceptable excipients. The term “excipient” herein means any substance, not itself a therapeutic agent, used as a carrier or vehicle for delivery of a therapeutic agent to a subject or added to a pharmaceutical composition to improve its handling or storage properties or to permit or facilitate formation of a unit dose of the composition.

Illustrative excipients include antioxidants, surfactants, adhesives, agents to adjust the pH and osmolarity, preservatives, thickening agents, sweetening agents, flavoring agents, taste masking agents, colorants, buffering agents, and penetration enhancers. Generally speaking, a given excipient, if present, will be present in an amount of about 0.001% to about 95%, about 0.01% to about 80%, about 0.02% to about 25%, or about 0.3% to about 10%, by weight.

Illustrative antioxidants for use in the present invention include, but are not limited to, butylated hydroxytoluene, butylated hydroxyanisole, potassium metabisulfite, and the like. One or more antioxidants, if desired, are typically present in a composition of the invention in an amount of about 0.01% to about 2.5%, for example about 0.01%, about 0.05%, about 0.1%, about 0.5%, about 1%, about 1.5%, about 1.75%, about 2%, about 2.25%, or about 2.5%, by weight.

In various embodiments, compositions of the invention comprise a preservative. Ideally, the optional preservative will be present in quantities sufficient to preserve the composition, but in quantities low enough that they do not cause irritation of the nasal mucosa. Suitable preservatives include, but are not limited to, benzalkonium chloride, methyl, ethyl, propyl or butylparaben, benzyl alcohol, phenylethyl alcohol, benzethonium, or combination thereof. Typically, the optional preservative is present in an amount of about 0.01% to about 0.5% or about 0.01% to about 2.5%, by weight.

In other embodiments, compositions of the invention are preservative-free. As used herein, the term “preservative-free” includes compositions that do not contain any preservative. Thus, in various embodiments, the composition does not contain, for example, benzalkonium chloride, methyl, ethyl, propyl or butylparaben, benzyl alcohol, phenylethyl alcohol, or benzethonium.

In one embodiment, compositions of the invention optionally comprise a buffering agent. The optional buffering agent, if present, is present in a composition of the invention in an amount that does not irritate the nasal mucosa. Buffering agents include agents that reduce pH changes. Illustrative classes of buffering agents for use in various embodiments of the present invention comprise a salt of a Group IA metal including, for example, a bicarbonate salt of a Group IA metal, a carbonate salt of a Group IA metal, an alkaline or alkali earth metal buffering agent, an aluminum buffering agent, a calcium buffering agent, a sodium buffering agent, or a magnesium buffering agent. Suitable buffering agents include carbonates, phosphates, bicarbonates, citrates, borates, acetates, phthalates, tartrates, succinates of any of the foregoing, for example sodium or potassium phosphate, citrate, borate, acetate, bicarbonate and carbonate.

Non-limiting examples of suitable buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). Furthermore, combinations or mixtures of any two or more of the above mentioned buffering agents can be used in the pharmaceutical compositions described herein. One or more buffering agents, if desired, are present in compositions of the invention in an amount of about 0.01% to about 5% or about 0.01% to about 3%, by weight.

In one embodiment, compositions of the invention optionally comprise one or more surfactants. Optional surfactants are typically present in a composition of the invention in an amount of about 0.1 mg/ml to about 10 mg/ml, about 0.5 mg/ml to 5 mg/ml or about 1 mg/ml.

In various embodiments, compositions the invention may include one or more agents that increase viscosity. Illustrative agents that increase viscosity include, but are not limited to, methylcellulose, carboxymethylcellulose sodium, ethylcellulose, carrageenan, carbopol, and/or combinations thereof. Typically, one or more viscosity increasing agents, if desired, are present in compositions of the invention in an amount of about 0.1% to about 10%, or about 0.1% to about 5%, by weight.

In various embodiments, compositions of the invention comprise one or more sweeteners and/or flavoring agents. Suitable sweeteners and/or flavoring agents include any agent that sweetens or provides flavor to a pharmaceutical composition. The sweetener or flavoring agent will help mask any bitter or bad taste that may occur if the pharmaceutical composition drips back into the mouth after intranasal administration. By addition of a sweetener or flavoring agent to the intranasal composition, a barrier that a patient may have to taking the intranasal composition because of unpleasant taste can be reduced. Optional sweetening agents and/or flavoring agents are typically present in a composition of the invention in an amount of about 0.1 mg/ml to about 10 mg/ml, about 0.5 mg/ml to 5 mg/ml or about 1 mg/ml.

Illustrative sweeteners or flavoring agents include, without limitation, acacia syrup, anethole, anise oil, aromatic elixir, benzaldehyde, benzaldehyde elixir, cyclodextrins, compound, caraway, caraway oil, cardamom oil, cardamom seed, cardamom spirit, compound, cardamom tincture, compound, cherry juice, cherry syrup, cinnamon, cinnamon oil, cinnamon water, citric acid, citric acid syrup, clove oil, cocoa, cocoa syrup, coriander oil, dextrose, eriodictyon, eriodictyon fluidextract, eriodictyon syrup, aromatic, ethylacetate, ethyl vanillin, fennel oil, ginger, ginger fluidextract, ginger oleoresin, dextrose, glucose, sugar, maltodextrin, glycerin, glycyrrhiza, glycyrrhiza elixir, glycyrrhiza extract, glycyrrhiza extract pure, glycyrrhiza fluidextract, glycyrrhiza syrup, honey, iso-alcoholic elixir, lavender oil, lemon oil, lemon tincture, mannitol, methyl salicylate, nutmeg oil, orange bitter, elixir, orange bitter, oil, orange flower oil, orange flower water, orange oil, orange peel, bitter, orange peel sweet, tincture, orange spirit, compound, orange syrup, peppermint, peppermint oil, peppermint spirit, peppermint water, phenylethyl alcohol, raspberry juice, raspberry syrup, rosemary oil, rose oil, rose water, stronger, saccharin, saccharin calcium, saccharin sodium, sarsaparilla syrup, sarsaparilla compound, sorbitol solution, spearmint, spearmint oil, sucrose, sucralose, syrup, thyme oil, tolu balsam, tolu balsam syrup, vanilla, vanilla tincture, vanillin, wild cherry syrup, or combinations thereof.

Illustrative taste masking agents includes, but are not limited to, cyclodextrins, cyclodextrins emulsions, cyclodextrins particles, cyclodextrins complexes, or combinations thereof.

The foregoing excipients can have multiple roles as is known in the art. For example, some flavoring agents can serve as sweeteners as well as a flavoring agent. Therefore, classification of excipients above is not to be construed as limiting in any manner.

Pharmaceutical compositions as disclosed herein are not limited to any particular pH. In one embodiment, pH of a composition of the invention ranges from about 2 to about 8, about 3 to about 6, or about 4 to about 6, for example about 5. If adjustment of pH is needed, it can be achieved by the addition of an appropriate acid, such as hydrochloric acid, or base, such as for example, sodium hydroxide.

Pharmaceutical compositions of the invention can be prepared in any suitable manner. In one embodiment, the compositions are prepared by mixing, in any order, a melatonin agonist with a liquid nasal carrier and one or more optional excipients at room temperature under aseptic conditions. In other embodiments, the mixture can be prepared under non-aseptic conditions and then sterile filtered, autoclaved or otherwise sterilized and packaged in a delivery device. It will be understood by those of ordinary skill in the art that the order of mixing is not critical, and the present invention includes without limitation mixing of compositions of the invention in any order.

### Stability

In one embodiment, a composition of the invention comprises at least about 85%, at least about 87%, at least about 90%, at least about 92%, at least about 95%, at least about 97%, or at least about 99% of the original melatonin agonist after storage (closed or open vessel) at 40° C. and 75% relative humidity for a period of at least about 1 week, at least about 2 weeks, at least about 3 weeks, at least about 4 weeks, at least about 6 weeks, at least about 8 weeks, at least about 10 weeks, at least about 15 weeks, at least about 20 weeks, at least about 25 weeks, at least about 30 weeks, at least about 35 weeks, at least about 40 weeks, at least about 45 weeks, or at least about 50 weeks.

### Method of Treatment

Compositions of the invention are useful in the treatment and/or prevention of, inter alia, a circadian rhythm disorders, sleep-awake rhythm disorders, time zone change syndrome, jet lag, sleep disorders, etc., or any other melatonin-mediated disease or disorder.

In one embodiment, the present invention provides a method for treating and/or preventing any of the above disorders in a subject in need thereof comprising intranasally administering to a subject a composition as described herein. The intranasal administration can occur about 1 to 30, about 1 to about 20, about 1 to about 10 or about 1 to about 5 times per day, per week, or per month.

In one embodiment, compositions of the invention are administered to a subject in an amount of about 0.0005 to about 2 mg/kg body weight, about 0.001 mg/kg body weight, about 0.001 to about 0.5 mg/kg body weight.

### Delivery Device

Compositions of the present invention can be administered using any suitable intranasal delivery device. In one embodiment, the delivery device is a unit-dose delivery device. Delivery devices comprising any of the pharmaceutical compositions of various embodiments disclosed herein comprise further embodiments of the invention. Non-limiting examples of suitable intranasal delivery devices, or components thereof, are disclosed in the following U.S. patents and U.S. patent publications, each of which are hereby incorporated by reference herein in their entirety: U.S. Pat. No. 4,946,069; U.S. Pat. No. 5,307,953; U.S. Pat. No. 5,368,201; U.S. Pat. No. 5,395,032; U.S. Pat. No. 5,427,280; U.S. Pat. No. 5,482,193; U.S. Pat. No. 5,584,417; U.S. Pat. No. 5,813,570; U.S. Pat. No. 5,893,484; U.S. Pat. No. 5,944,222; U.S. Pat. No. 5,964,417; U.S. Pat. No. 5,967,369; U.S. Pat. No. 6,062,433; U.S. Pat. No. 6,257,454; U.S. Pat. No. 6,626,379; U.S. Pat. No. 6,321,942; U.S. Pat. No. 6,367,473; and U.S. Pat. No. 6,948,492.

The delivery device can be filled with single or multidose amounts of melatonin agonists. In one embodiment, the invention provides a vessel or container holding the pharmaceutical composition; any optional sealing means are sterilizable. In one such embodiment, the parts of the device that are in contact with the pharmaceutical composition can be constructed and assembled in a configuration so as to allow for sterilization. Devices with one or more unit-dose(s) can be sterilized either before or after filling and/or packaging, employing methods and technology that are well known in the art. Individual devices can be packaged, sterilized and shipped; alternatively, entire shipping and storage packages can be sterilized at once, and the devices removed individually for dispensing, without affecting the sterility of the remaining units.

In one embodiment, the volume of liquid contained in each vessel of a delivery device is about 0.025 ml to about 2 ml, about 0.25 ml to 1 ml, or about 0.05 ml to about 0.15 ml.

In another embodiment, a composition of the invention, upon being discharged from an intranasal spray device at a spray distance of 1 cm from a detection laser, for example at a discharge volume of about 100 μl per spray, exhibits a droplet size distribution having a mean Dv10 of about 5 to about 50 μm, about 7.5 to about 40 μm, or about 10 to about 35 μm; a mean Dv50 of about 15 to about 80 μm, about 20 to about 70 μm, or about 30 to about 60 μm; and/or a mean Dv90 of about 40 to about 130 μm, about 50 to about 120 μm, or about 60 to about 100 μm. In another embodiment, the spray has a mean span [(Dv90−Dv10/Dv50)] of about 1 to about 5, about 1.25 to about 4, or about 1.5 to about 3.

In a related embodiment, upon positioning the device 1 cm away from an impaction plate, actuating the device to produce a spray pattern onto the impaction plate, and measuring the diameter of the spray pattern the spray pattern has a maximum diameter (Dₘₐₓ) of about 1 to about 4 cm, about 2 to about 3 cm or about 2.2 to about 2.5 cm, for example about 2.3 cm. In another related embodiment, the spray has a minimum diameter (Dₘᵢₙ) of about 1 to about 3 cm, about 1.5 to about 2.8 cm or about 1.8 to about 2.3 cm, for example about 2.1 cm.

### Incorporation by Reference

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respect illustrative rather than limiting the invention described herein. Scope of the invention is thus indicated by the appended claims rather than by the foregoing description, and all changes that come with in the meaning and range of equivalency of the claims are intended to be embraced therein.

We claim:

1. An intranasally deliverable pharmaceutical composition comprising a therapeutically effective amount of a tricyclic melatonin receptor agonist or pharmaceutically acceptable salt thereof and a liquid nasal carrier, wherein at least a portion of the agonist or salt thereof is in dissolved or solubilized form in the carrier.

2. The composition of claim 1, wherein the melatonin agonist comprises a compound of Formula 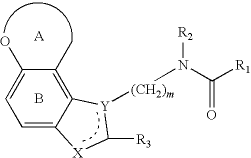
wherein,
R¹ represents an optionally substituted hydrocarbon group, an optionally substituted amino group or an optionally substituted heterocyclic group;
R² represents a hydrogen atom or an optionally substituted hydrocarbon group;
R³ represents a hydrogen atom, an optionally substituted hydrocarbon group or an optionally substituted heterocyclic group;
X represents CHR⁴, CR⁴, N, NR⁴, O or S;
R⁴ represents a hydrogen atom or an optionally substituted hydrocarbon group;
Y represents C, CH or N, provided that when X is CH₂, Y is C or CH; — [symbol]
independently represents a single bond or a double bond;
ring A represents an optionally substituted, 5- to 7-membered oxygen-containing heterocyclic ring;
ring B represents an optionally substituted benzene ring; and
m represents an integer of 1 to 4; or a pharmaceutically acceptable salt thereof.

3. The composition of claim 2, wherein the liquid nasal carrier comprises water.

4. The composition of claim 3, wherein the liquid nasal carrier further comprises at least one pharmaceutically acceptable solvent or co-solvent.

5. The composition of claim 1, wherein R¹ is an optionally substituted C₁₋₆ alkyl group, an optionally substituted C₃₋₆ cycloalkyl group, an optionally substituted C₂₋₆ alkenyl group, an optionally substituted C₆₋₁₄ aryl group, an optionally substituted mono- or di-C₁₋₆ alkylamino group, an optionally substituted C₆₋₁₄ arylamino group, or an optionally substituted 5- or 6-membered nitrogen-containing heterocyclic group.

6. The composition of claim 5, wherein R¹ is a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkylcarbamoyl, di-C₁₋₆ alkylcarbamoyl, C₆₋₁₀ aryl-carbamoyl, C₆₋₁₀ aryl, C₆₋₁₀ aryloxy and an optionally halogenated C₁₋₆ alkyl-carbonylamino.

7. The composition of claim 5, wherein R¹ is an amino group which may be substituted by 1 or 2 substituents selected from the group consisting of a C₁₋₆ alkyl, C₂₋₆ alkenyl, C₂₋₆ alkynyl, C₃₋₆ cycloalkyl and C₆₋₁₄ aryl group, each of which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino.

8. The composition of claim 5, wherein R¹ is a 5- to 14-membered heterocyclic group containing, besides carbon atoms, 1 to 3 hetero atoms selected from nitrogen atom, oxygen atom and sulfur atom, which group may be substituted by 1 to 5 substituents selected from the group consisting of a halogen, C₁₋₆ alkyl, C₃₋₆ cycloalkyl, C₂₋₆ alkynyl, C₂₋₆ alkenyl, C₇₋₁₁ aralkyl, C₆₋₁₀ aryl, C₁₋₆ alkoxy, C₆₋₁₀ aryloxy, formyl, C₁₋₆ alkyl-carbonyl, C₆₋₁₀ aryl-carbonyl, formyloxy, C₁₋₆ alkylcarbonyloxy, C₆₋₁₀ aryl-carbonyloxy, carboxyl, C₁₋₆ alkoxy-carbonyl, C₇₋₁₁ aralkyloxy-carbonyl, carbamoyl, an optionally halogenated C₁₋₄ alkyl, oxo, amidino, imino, amino, mono-C₁₋₄ alkylamino, di-C₁₋₄ alkylamino, 3- to 6-membered cyclic amino, C₁₋₃ alkylenedioxy, hydroxy, nitro, cyano, mercapto, sulfo, sulfino, phosphono, sulfamoyl, mono-C₁₋₆ alkylsulfamoyl, di-C₁₋₆ alkylsulfamoyl, C₁₋₆ alkylthio, C₆₋₁₀ arylthio, C₁₋₆ alkylsulfinyl, C₆₋₁₀ arylsulfinyl, C₁₋₆ alkylsulfonyl and C₆₋₁₀ arylsulfonyl.

9. The composition of claim 5, wherein R² is selected from (i) a hydrogen atom or (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonyl amino.

10. The composition of claim 5, wherein R³ is selected from (i) a hydrogen atom, (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino or (iii) a 5- to 14-membered heterocyclic group containing, besides carbon atoms, 1 to 3 hetero atoms selected from nitrogen atom, oxygen atom and sulfur atom, which group may be substituted by 1 to 5 substituents selected from the group consisting of a halogen, C₁₋₆ alkyl, C₃₋₆ cycloalkyl, C₂₋₆ alkynyl, C₂₋₆ alkenyl, C₇₋₁₁ aralkyl, C₆₋₁₀ aryl, C₁₋₆ alkoxy, C₆₋₁₀ aryloxy, formyl, C₁₋₆ alkyl-carbonyl, C₆₋₁₀ aryl-carbonyl, formyloxy, C₁₋₆ alkylcarbonyloxy, C₆₋₁₀ aryl-carbonyloxy, carboxyl, C₁₋₆ alkoxy-carbonyl, C₇₋₁₁ aralkyloxy-carbonyl, carbamoyl, an optionally halogenated C₁₋₄ alkyl, oxo, amidino, imino, amino, mono-C₁₋₄ alkylamino, di-C₁₋₄ alkylamino, 3- to 6-membered cyclic amino, C₁₋₃ alkylenedioxy, hydroxy, nitro, cyano, mercapto, sulfo, sulfino, phosphono, sulfamoyl, mono-C₁₋₆ alkylsulfamoyl, di-C₁₋₆ alkylsulfamoyl, C₁₋₆ alkylthio, C₆₋₁₀ arylthio, C₁₋₆ alkylsulfinyl, C₆₋₁₀ arylsulfinyl, C₁₋₆ alkylsulfonyl and C₆₋₁₀ arylsulfonyl.

11. The composition of claim 5, wherein R⁴ is selected from (i) a hydrogen atom or (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino.

12. The composition of claim 5, wherein ring A is a 5- to 7-membered heterocyclic group optionally containing, besides carbon atoms and an oxygen atom, 1 to 3 hetero atoms selected from nitrogen atom, oxygen atom and sulfur atom, which group may be substituted by 1 to 4 substituents selected from the group consisting of (i) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino, (ii) a halogen, (iii) C₁₋₆ alkoxy, (iv) C₆₋₁₀ aryloxy, (v) formyl, (vi) C₁₋₆ alkyl-carbonyl, (vii) C₆₋₁₀ aryl-carbonyl, (viii) formyloxy, (ix) C₁₋₆ alkyl-carbonyloxy, (x) C₆₋₁₀ aryl-carbonyloxy, (xi) carboxyl, (xii) C₁₋₆ alkoxy-carbonyl, (xiii) C₇₋₁₁ aralkyloxy-carbonyl, (xiv) carbamoyl, (xv) an optionally halogenated C₁₋₄ alkyl, (xvi) oxo, (xvii) amidino, (xviii) imino, (xix) amino, (xx) mono-C₁₋₄ alkylamino, (xxi) di-C₁₋₄ alkylamino, (xxii) 3- to 6-membered cyclic amino, (xxiii) C₁₋₃ alkylenedioxy, (xxiv) hydroxy, (xxv) nitro, (xxvi) cyano, (xxvii) mercapto, (xxviii) sulfo, (xxix) sulfino, (xxx) phosphono, (xxxi) sulfamoyl, (xxxii) mono-C₁₋₆ alkylsulfamoyl, (xxxiii) di-C₁₋₆ alkylsulfamoyl, (xxxiv) C₁₋₆ alkylthio, (xxxv) C₆₋₁₀ arylthio, (xxxvi) C₁₋₆ alkylsulfinyl, (xxxvii) C₆₋₁₀ arylsulfinyl, (xxxviii) C₁₋₆ alkylsulfonyl and (xxxix) C₆₋₁₀ arylsulfonyl.

13. The composition of claim 5, wherein ring B is a benzene ring which may be substituted by 1 or 2 substituents selected from the group consisting of (i) a halogen, (ii) a C₁₋₆ alkyl, C₂₋₆ alkenyl, C₂₋₆ alkynyl, C₃₋₆ cycloalkyl or C₆₋₁₄ aryl group which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino, (iii) an amino group which may be substituted by 1 or 2 substituents selected from the group consisting of a C₁₋₆ alkyl, C₂₋₆ alkenyl, alkynyl, C₃₋₆ cycloalkyl and C₆₋₁₄ aryl group, each of which may be substituted by 1 to 5 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkylcarbamoyl, C₆₋₁₀ aryl-carbamoyl, C₆₋₁₀ aryl, C₆₋₁₀-aryloxy and an optionally halogenated C₁₋₆ alkyl-carbonylamino, (iv) a C₁₋₆ alkanoylamino group, (v) a C₁₋₆ alkoxy group which may be substituted by 1 to 3 substituents selected from the group consisting of a halogen, nitro, cyano, hydroxy, an optionally halogenated C₁₋₆ alkyl, C₁₋₆ alkoxy, amino, mono-C₁₋₆ alkylamino, di-C₁₋₆ alkylamino, carboxyl, C₁₋₆ alkyl-carbonyl, C₁₋₆ alkoxy-carbonyl, carbamoyl, mono-C₁₋₆ alkyl-carbamoyl, di-C₁₋₆ alkyl-carbamoyl, C₆₋₁₀ aryl-carbamoyl, C₆₋₁₀ aryl, C₆₋₁₀ aryloxy and an optionally halogenated C₁₋₆ alkyl-carbonylamino or (vi) a C₁₋₃ alkylenedioxy group.

14. The composition of claim 1, wherein the melatonin receptor agonist is selected from the group consisting of:
N-[2-(1,6,7,8-tetrahydro-2H-indeno[5,4-b]furan-8-yl)ethyl]acetamide;
N-[2-(1,6,7,8-tetrahydro-2H-indeno[5,4-b]furan-8-yl)ethyl]butyramide;
N-[2-(1,6,7,8-tetrahydro-2H-indeno[5,4-b]furan-8-yl)ethyl]propionamide;
N-[2-(3,7,8,9-tetrahydropyrano[3,2-e]indol-1-yl)ethyl]propionamide;
N-[2-(3,7,8,9-tetrahydropyrano[3,2-e]indol-1-yl)ethyl]butyramide;
N-[2-(1,2,3,7,8,9-hexahydropyrano[3,2-e]indol-1-yl)ethyl]propionamide;
N-[2-(1,2,3,7,8,9-hexahydropyrano[3,2-e]indol-1-yl)ethyl]butyramide;
N-[2-(4-fluoro-1,6,7,8-tetrahydro-2H-indeno[5,4-b]furan-8-yl)ethyl]butyramide;
N-[2-(4-fluoro-1,6,7,8-tetrahydro-2H-indeno[5,4-b]furan-8-yl)ethyl]propionamide;
N-[2-(5-fluoro-3,7,8,9-tetrahydrocyclopenta[f][1]benzopyran-9-yl)ethyl]propionamide;
(S)—N-[2-(1,6,7,8-tetrahydro-2H-indeno[5,4-b]furan-8-yl)ethyl]propionamide;
(R)—N-[2-(1,6,7,8-tetrahydro-2H-indeno[5,4-b]furan-8-yl)ethyl]propionamide;
N-[2-(1,6,7,8-tetrahydro-2H-indeno[5,4-b]furan-8-yl)ethyl)]butyramide;
N-[2-(1,6-dihydro-2H-indeno[5,4-b]furan-8-yl)ethyl]acetamide;
N-[2-(1,6-dihydro-2H-indeno[5,4-b]furan-8-yl)ethyl]propionamide;
N-[2-(1,6-dihydro-2H-indeno[5,4-b]furan-8-yl)ethyl]butyramide;
N-[2-(7,8-dihydro-6H-indeno[4,5-d]-1,3-dioxol-8-yl)ethyl]propionamide;
N-[2-(7,8-dihydro-6H-indeno[4,5-d]-1,3-dioxol-8-yl)ethyl]butyramide;
N-[2-(2,3,8,9-tetrahydro-7H-indeno[4,5-b]-1,4-dioxyn-9-yl)ethyl]propionamide;
N-[2-(2,3,8,9-tetrahydro-7H-indeno[4,5-b]-1,4-dioxyn-9-yl)ethyl]butyramide;
N-[2-(1,6,7,8-tetrahydro-2H-furo[3,2-e]indol-8-yl)ethyl]propionamide;
N-[2-(1,6,7,8-tetrahydro-2H-furo[3,2-e]indol-8-yl)ethyl]butyramide;
N-[2-(7-phenyl-1,6-dihydro-2H-indeno[5,4-b]furan-8-yl)ethyl]propionamide;
N-[2-(7-phenyl-1,6-dihydro-2H-indeno[5,4-b]furan-8-yl)ethyl]butyramide; or a pharmaceutically acceptable salt of any of the foregoing.

15. The composition of claim 1, wherein the melatonin agonist is 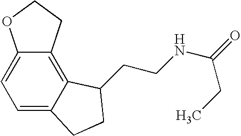
or a pharmaceutically acceptable salt thereof.

16. The composition of claim 4, wherein the at least one solvent or co-solvent is selected from propylene glycol, alcohol, glycerol, isopropylalcohol and polyethylene glycol and combinations thereof.

17. A method of treating a disease or disorder where treatment with a melatonin receptor agonist is indicated, the method comprising intranasally administering to a subject in need thereof a therapeutically effective amount of a composition of claim 1.

18. The method of claim 17, wherein the subject suffers from a melatonin-mediated disease or disorder.

19. The method of claim 18, wherein the melatonin-mediated disease or disorder is a circadian rhythm disorder, a sleep-awake rhythm disorder, a time zone change syndrome, a jet lag-related disorder or other sleep disorder.
